(19) United States
(12) Patent Application Publication
(10) Pub. No.: US 2006/0271172 A1
(43) Pub. Date: Nov. 30, 2006

(54) Minimally Invasive Aortic Valve Replacement

(76) Inventor: Hassan Tehrani, Miami Beach, FL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/383,578

(22) Filed: May 16, 2006

Related U.S. Application Data
(60) Provisional application No. 60/681,306, filed on May 16, 2005.

(51) Int. Cl. A61F 2/24

(52) U.S. Cl. 623/2.11; 623/2.38

(57) ABSTRACT

A replacement cardiac valve for placement adjacent the native annulus comprises a valve body having a multi-leaflet valve, a supporting stent surrounding and operatively coupled to the valve body, a superior O-ring and an inferior O-ring spaced from one another to span the native annulus, the O-rings surrounding the valve body and operatively coupled the valve body or the supporting stent, the valve body, the supporting stent, and the O-rings adapted for transcatheter placement using a deployment catheter.

### RELATED APPLICATIONS

This application claims priority under 35 U.S.C. § 119(e) from U.S. provisional application Ser. No. 60/681,306, filed May 16, 2005.

### FIELD OF THE INVENTION

The present invention relates generally to the minimally invasive replacement of aortic valves.

### BACKGROUND OF THE INVENTION

In 1998 alone, in the United States approximately 80,000 valve procedures were performed. When indicated, the treatment of aortic valve disease for stenosis or regurgitation typically has been limited to valve replacement.

Conventional valve replacement necessitates median stemotomy and institution of cardiopulmonary bypass. Elective aortic valve surgery in appropriately selected patients may have mortality rates as low as 4%. However this mortality rate rises to 13% in an urgent or emergent setting. Combined major and minor morbidity may be as high as 40%, even for elective valve replacement.

In recent years, a number of researchers have investigated the feasibility of minimally invasive aortic valve replacement. Minimally invasive aortic valve replacement can involve either inserting a new valve through a relatively small incision in the chest while on cardiopulmonary bypass, or can involve inserting the new valve using transcatheter techniques. This latter technique, which may avoid cardiopulmonary bypass altogether, involves using balloon catheters to initially perform a valvuloplasty to open and push the diseased valve leaflets aside. The valvuloplasty is followed by deployment of the percutaneous valve inside the native valve, using remote catheter-based techniques, which are typically performed under fluoroscopic or ultrasound guidance.

Conventional new or replacement aortic valves are typically hand sewn into place using multiple sutures that fix the sewing ring of the new valve to the native aortic annuls after the old valve has been excised. A limiting factor for inserting the new valve through a smaller incision is the ability to place all of the sutures through the native aortic annulus.

Percutaneous aortic valve designs that are currently being investigated may suffer certain limitations. A major issue has been the potential for the lack of a lack of fixation and seal between the percutaneous valve and the native valve annulus. The possible resulting leakage is termed a paravalvular leak (FIG. 1). In this circumstance, despite the presence of a structurally intact valve, the function of the entire valve apparatus between the left ventricle of the heart and the ascending aorta is compromised, which can result in heart failure. Proper fixation is an important consideration, as improper fixation can, in certain circumstances, lead to migration or embolization of the valve.

### SUMMARY OF THE INVENTION

In accordance with an aspect of the invention, a replacement cardiac valve for minimally-invasive placement comprises a valve body having a valve leaflet apparatus and a supporting stent, and an O-ring assembly surrounding the valve body and including a superior O-ring and an inferior O-ring. The valve body and the O-ring assembly are adapted for transcatheter placement using a deployment catheter.

In further accordance with a disclosed example, the superior and inferior O-rings are spaced to span the native valve annulus, with the O-rings preferably constructed of felt. The valve body may comprise a multi-leaflet valve, such as a bi-leaflet or tri-leaflet valve. The valve may be constructed of at least one of expanded polytetrafluoroethylene (ePTFE), bovine pericardium, or native porcine valve leaflets, and the O-rings may be constructed of expanded polytetrafluoroethylene, foam, or rubber.

In accordance with another aspect of the invention, a replacement cardiac valve for minimally-invasive placement adjacent the native annulus comprises a valve body having a multi-leaflet valve and a supporting stent, a superior O-ring and an inferior O-ring, with both O-rings operatively coupled to the valve body and spaced to span the native annulus, and with the valve body and the superior and inferior O-rings adapted for transcatheter placement using a deployment catheter.

In accordance with a further aspect of the invention, a replacement cardiac valve for placement adjacent the native annulus comprises a valve body having a multi-leaflet valve, a supporting stent surrounding the valve body, the valve body and the supporting stent operatively coupled to one another, a superior O-ring and an inferior O-ring, with both O-rings surrounding the valve body and operatively coupled the valve body or the supporting stent, and with the O-rings spaced apart a distance sufficient to span the native annulus. The valve body, the supporting stent, and the O-rings all are adapted for transcatheter placement using a deployment catheter.

In accordance with yet a further aspect of the invention, a method of minimally-invasive valve replacement at a location adjacent a native annulus comprises the steps of performing a balloon valvuloplasty on a native valve, providing a replacement valve having a pair of O-rings surrounding a valve body, guiding the replacement valve to a position adjacent the valve annulus of the native valve, expanding the valve body at a positioned adjacent the valve annulus of the native valve, and forming a paravalvular seal.

### BRIEF DESCRIPTION OF THE DRAWINGS

FIG. 1 depicts a percutaneous valve and a native valve and includes an arrow demonstrating the area of a typical paravalvular leak.

FIG. 2 is a depiction of a valvuloplasty ballon, a balloon catheter, and an umbrella filter.

FIG. 3A is an end view of a minimally-invasive replacement valve having an outer supporting stent and a pair of exemplary valve leaflets.

FIG. 3B is an end view of a minimally-invasive replacement valve having an outer supporting stent and three exemplary valve leaflets.

FIG. 4 is a perspective view illustrating the exterior of the minimally-invasive replacement valve surrounded by superior and inferior O-rings and adapted for placement in accordance with the teachings of the present invention.

FIG. 5 illustrates a stent valve crimped and mounted on a balloon deployment catheter.

FIG. 6 is an enlarged fragmentary cross-sectional illustrating the replacement valve of FIG. 3A or 3B positioned within an aortic vessel adjacent the native valve annulus.

### DETAILED DESCRIPTION

Although the following text sets forth a detailed description of exemplary embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, with those alternative embodiments still falling within the scope of the claims defining the invention.

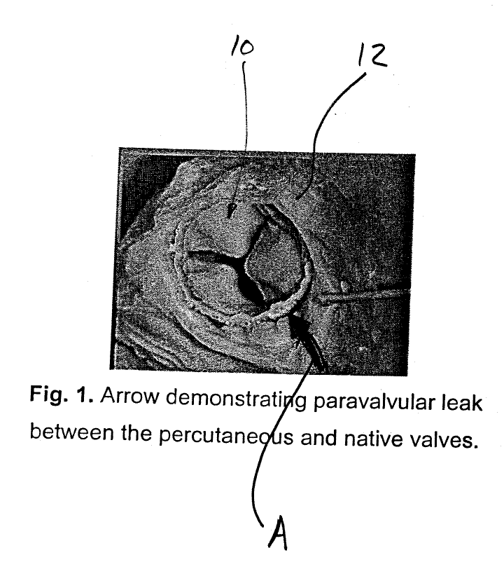

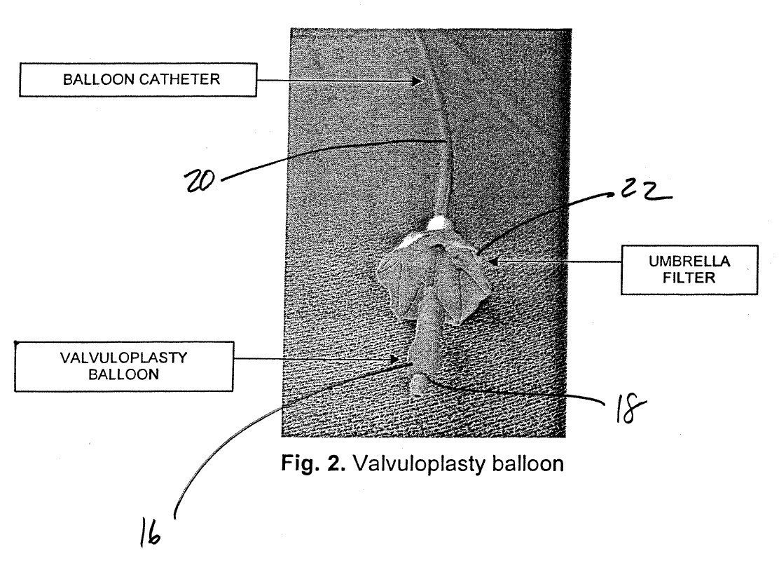

Referring now to the drawings, FIG. 1 illustrates a percutaneous valve 10 disposed inside of an aortic vessel 12. A paravalvular leak is indicated by the reference arrow A between the percutaneous valve 10 and the dilated native valve 14. FIG. 2 illustrates a valvuloplasty balloon 16 attached adjacent an end 18 of a balloon catheter 20. The balloon catheter 20 includes an umbrella filter 22. The balloon catheter 20 and its associated component parts may be conventional and of the type commonly employed in percutaneous cardiac operations.

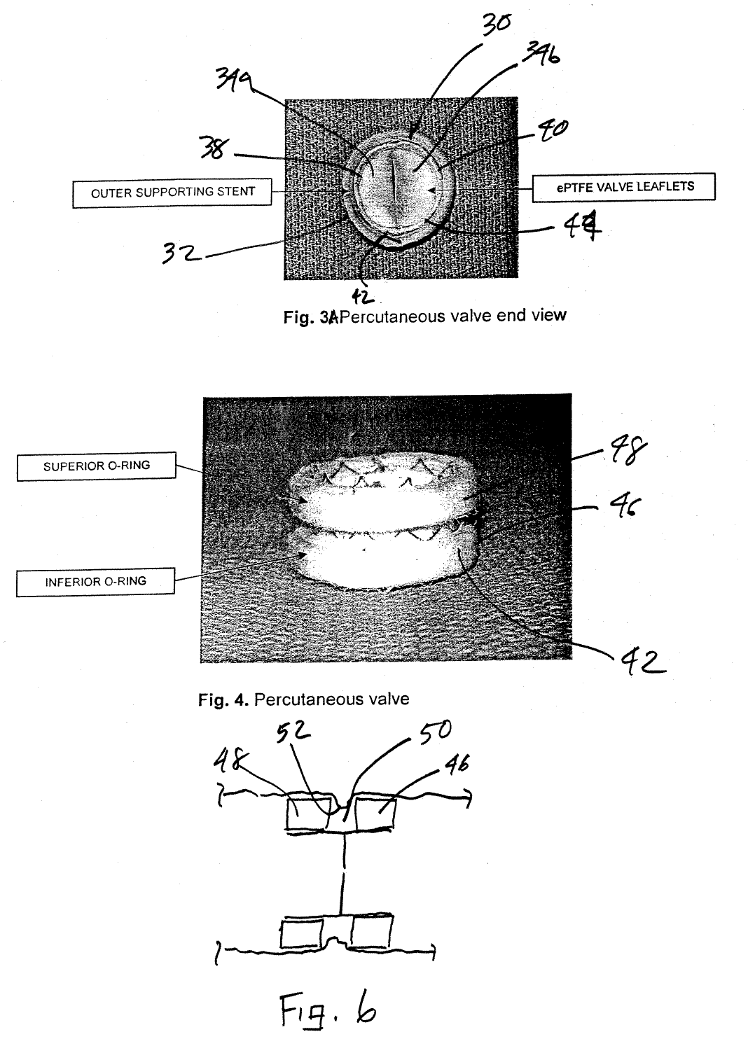

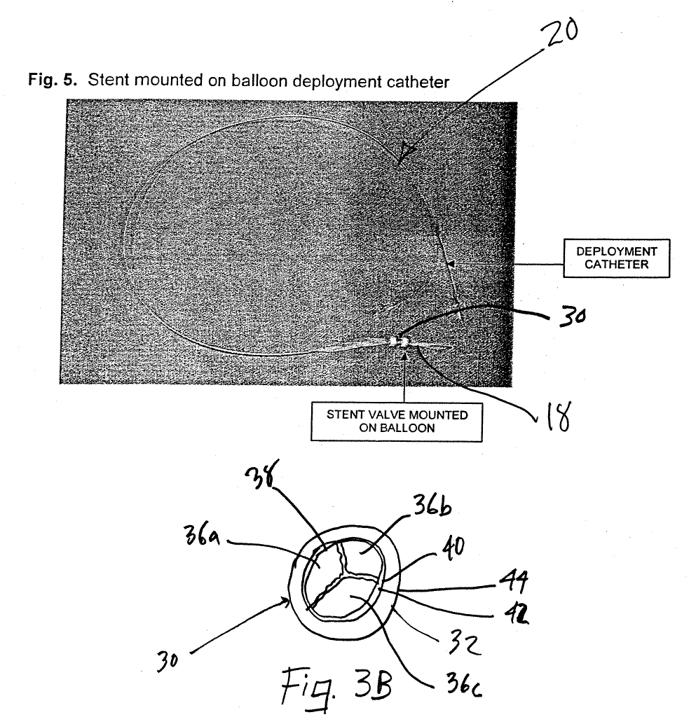

FIGS. 3A and 3B illustrate a minimal a invasive replacement valve 30 assembled in accordance with the teachings of a first disclosed example of the present invention (FIG. 3A) and a second disclosed example of the present invention (FIG. 3B). While both examples illustrate multi-leaflet valves 32, the replacement valve 30 illustrated in FIG. 3A is a bi-leaflet valve having a pair of leaflets 34a and 34b. The replacement valve 30 illustrated in FIG. 3B is a tri-leaflet valve having leaflets 36a, 36b, and 36c. For ease of reference, the following discussion will make reference only to a replacement valve 30, it being understood that the replacement valve 30 may comprise the bi-leaflet valve of FIG. 3A or the tri-leaflet valve of FIG. 3B. Other replacement valve leaflet arrangements made prove suitable for use with further details of the replacement valve to be discussed below.

The replacement valve 30 includes a valve body 38 having the valve leaflets 34a, 34b, or 36a-36c, joined to an outer periphery 40. The valve leaflets may be constructed of, for example, expanded polytetrafluoroethylene (ePTFE), bovine pericardium, or native porcine valve leaflets. Other materials may prove suitable. The valve body 38 is surrounded by and O-ring assembly 42 and a supporting stent 44 (the supporting stent 44 is best visible in FIGS. 4 and 6).

Referring now to FIG. 4, the O-ring assembly 42 generally surrounds the supporting stent 44 as well as the valve body 38. The O-ring assembly 42 and inferior O-ring 46 and a superior O-ring 48. In accordance with the disclosed example, both of the O-rings preferably are constructed of felt, although other materials may prove suitable. Possible other suitable materials include, by way of example rather than limitation, polytetrafluoroethylene, foam, or rubber. The supporting stent 44 may be conventional. The outer periphery of the valve body is preferably joined to the interior of the stent 44 using any conventional means. The inferior and superior O-rings 46 and 48 preferably are joined about the supporting stent 44 using, for example, an adhesive of the type commonly employed in the manufacture of replacement valves, or by any other attachment mechanism that proves suitable. Referring to FIG. 5, the replacement valve 30 is sized and shaped to be fitted over the balloon of the balloon catheter. Accordingly, it will be understood that the replacement valve 30 (and its associated components as outlined above) can be compressed from the expanded position shown in FIG. 4 to the position of FIG. 5 in which the replacement valve 30 is in a compressed position and is installed on the catheter over the balloon and is ready for deployment in the appropriate cardiac vessel 12.

Referring now to FIG. 6, the replacement valve 30 is shown in its expanded position inside the appropriate cardiac vessel 12. The O-ring assembly 42 is spaced apart to define a gap 50 that is sized to span the native valve annulus 52. In accordance with the disclosed example, the O-ring assembly 42 preferably minimizes or prevents valve migration, paravalvular leakage and embolization.

Further aspects or the disclosed example are explained in greater detail below.

I. Percutaneous Aortic Valve and Deployment Apparatus

An exemplary procedure consists of percutaneous balloon valvuloplasty followed by deployment of the valve with the native annulus. Further exemplary details of this apparatus are described below.

A. Valvuloplasty Balloon

The initial part of the procedure would be to perform a conventional balloon valvuloplasty. This procedure typically is performed via a femoral artery approach. As would be known, the valvuloplasty would break the native stenotic valve cusps, allowing them to be easily pushed back into the coronary sinuses when the percutaneous valve is deployed. Therefore, this procedure typically does not necessitate removal of the native valve apparatus. Instead, the valve cusps are simply pushed out of the way. In order to avoid the consequences of embolization, an umbrella shaped filter is mounted distal to the balloon and is opened prior to balloon inflation to catch any debris (FIG. 2).

B. Valve Design

Referring to FIG. 3A or 3B, a replacement valve 30 assembled in accordance with the teachings of a disclosed example of the present invention includes an outer supporting metallic stent, and an inner valve leaflet apparatus. The metallic stent preferably is constructed from nitinol or stainless steel, or from any other suitable material. The exemplary valve leaflets shown are constructed of expanded polytetrafluoroethylene (ePTFE). Alternatively, the leaflets may be constructed from bovine pericardium or native porcine valve leaflets similar to currently available bioprosthetic aortic valves. Other materials may prove suitable. In order to overcome or at least reduce the problem of paravalvular leaks, the outer supporting stent is encircled by two O-rings.

In accordance with a disclosed example, and as shown in FIG. 4, the O-rings are constructed of felt. Alternatively, the O-rings may be constructed from, for example, ePTFE, foam, rubber, or any other material that proves suitable. As a further alternative, the O-rings may be constructed of a hollow membrane and may be filled or inflated with a suitable material once the valve has been placed at the level of the aortic annulus. Preferably, maximal seal is obtained by positioning the valve so the gap 50 (the area between the superior and inferior O-rings 46 and 48) is disposed at the level of the aortic annulus 52, with the superior ring sitting just above the level of the annulus, and the inferior ring sitting just below the level of the annulus.

C. Valve Deployment

There are two presently contemplated methods for inserting the valve. In the first method, the patient is placed on cardiopulmonary bypass through the femoral vessels. A small incision is made on the upper sternum to access the ascending aorta. The aorta is clamped and opened to expose the diseased aortic valve which is excised. The new valve is then inserted under direct vision in such a manner that the mid-portion (the area or gap between the superior and inferior O-rings) is disposed at the level of the aortic annulus, with the superior ring sitting just above the level of the annulus, and the inferior ring sitting just below the level of the annulus. In accordance with the disclosed example, the O-rings assist in fixing the valve to the annulus and prevent paravalvular leak. Additional fixation of the O-ring to the annulus may be obtained by any currently available bioadhesive.

The second method involves the transcatheter approach. In this method the valve is collapsed or crimped onto a balloon catheter. Preferably, the valve is delivered preloaded on a balloon catheter. This balloon catheter typically is inserted via a peripheral artery approach, typically via the femoral artery. Conventionally, the deployment catheter is positioned under fluoroscopic or echocardiographic guidance into the native valve annulus. The valve is then deployed by expanding the balloon. Preferably, successful deployment is confirmed by radiographic and echocardiograhic techniques.

Numerous modifications and alternative embodiments of the invention will be apparent to those skilled in the art in view of the foregoing descriptions. Accordingly, these descriptions are to be construed as illustrative only and are for the purpose of teaching those skilled in the art the best mode or modes presently contemplated for carrying out the invention. The details of the structure or structures disclosed herein may be varied substantially without departing from the spirit of the invention, and the exclusive use of all modifications which come within the scope of the appended claims, either literally or under the doctrine of equivalents, is reserved.

What is claimed:

1. A replacement cardiac valve for minimally-invasive placement comprising:
a valve body having a valve leaflet apparatus and a supporting stent;
an O-ring assembly surrounding the valve body and including a superior O-ring and an inferior O-ring;
the valve body and the O-ring assembly adapted for transcatheter placement using a deployment catheter.

2. The cardiac valve of claim 1, wherein the superior and inferior O-rings are spaced to span the native valve annulus.

3. The cardiac valve of claim 1, wherein the superior and the inferior O-rings are constructed of felt.

4. The cardiac valve of claim 1, wherein the valve body is a bi-leaflet valve.

5. The cardiac valve of claim 1, wherein the valve body is a tri-leaflet valve.

6. The cardiac valve of claim 4, wherein the bi-leaflet valve is constructed of at least one of expanded polytetrafluoroethylene (ePTFE), bovine pericardium, or native porcine valve leaflets.

7. The cardiac valve of claim 5, wherein the tri-leaflet valve is constructed of at least one of expanded polytetrafluoroethylene (ePTFE), bovine pericardium, or native porcine valve leaflets.

8. The cardiac valve of claim 1, wherein the O-rings at constructed of expanded polytetrafluoroethylene, foam, or rubber.

9. A replacement cardiac valve for minimally-invasive placement adjacent the native annulus, the cardiac valve comprising:
a valve body having a multi-leaflet valve and a supporting stent;
a superior O-ring and an inferior O-ring, both O-rings operatively coupled to the valve body and spaced to span the native annulus;
the valve body and the superior and inferior O-rings adapted for transcatheter placement using a deployment catheter.

10. The cardiac valve of claim 9, wherein the superior and the inferior O-rings are constructed of felt.

11. The cardiac valve of claim 9, wherein the multi-leaflet valve is a bi-leaflet valve.

12. The device of claim 9, wherein the multi-leaflet valve is a tri-leaflet valve.

13. The device of claim 9, wherein the multi-leaflet valve is constructed of at least one of expanded polytetrafluoroethylene (ePTFE), bovine pericardium, or native porcine valve leaflets.

14. A replacement cardiac valve for placement adjacent the native annulus, the cardiac valve comprising:
a valve body having a multi-leaflet valve;
a supporting stent surrounding the valve body, the valve body and the supporting stent operatively coupled to one another;
a superior O-ring and an inferior O-ring, both O-rings surrounding the valve body and operatively coupled the valve body or the supporting stent, the O-rings spaced apart a distance sufficient to span the native annulus;
the valve body, the supporting stent, and the O-rings adapted for transcatheter placement using a deployment catheter.

15. The cardiac valve of claim 14, wherein the superior and the inferior O-rings are constructed of felt.

16. The cardiac valve of claim 14, wherein the multi-leaflet valve constructed of at least one of expanded polytetrafluoroethylene (ePTFE), bovine pericardium, or native porcine valve leaflets.

17. A method of minimally-invasive valve replacement at a location adjacent a native annulus, the method comprising the steps of:
performing a balloon valvuloplasty on a native valve;
providing a replacement valve having a pair of O-rings surrounding a valve body;
guiding the replacement valve to a position adjacent the valve annulus of the native valve;
expanding the valve body at a positioned adjacent the valve annulus of the native valve; and
forming a paravalvular seal.
